(19) United States
(12) Patent Application Publication
(10) Pub. No.: US 2022/0168589 A1
(43) Pub. Date: Jun. 2, 2022

(54) SKIN TREATMENT METHOD USING MEDICAL LASER, HAVING IMPROVED TREATMENT EFFICACY

(71) Applicant: LUTRONIC CORPORATION, Gyeonggi-do (KR)

(72) Inventors: Hee Chul LEE, Gyeonggi-do (KR); Ju Mi HONG, Gyeonggi-do (KR)

(21) Appl. No.: 17/593,559

(22) PCT Filed: Apr. 3, 2020
(86) PCT No.: PCT/KR2020/004574

§ 371 Date: Sep. 21, 2021

(30) Foreign Application Priority Data
Apr. 3, 2019 (KR) 10-2019-0039098

(51) Int. Cl. A61N 5/06; A61N 5/067

(52) U.S. Cl. CPC A61N 5/0616; A61N 5/067; A61N 2005/0629; A61N 2005/0659; A61N 2005/0663

(57) ABSTRACT

Provided area an operating method for a laser device for skin, having improved treatment efficacy, or a treatment method for improving skin conditions. In the method according to an aspect, energy generated from a medical laser beam may be selectively transferred to a specific treatment area, which may be implemented in one device. Therefore, the method may contribute to improving the efficacy of laser treatment.

### CROSS-REFERENCE TO RELATED APPLICATION

This application is a National Stage of International Application No. PCT/KR2020/004574, having an International Filing Date of 3 Apr. 2020, which designated the United States of America, and which International Application was published under PCT Article 21(2) as WO Publication No. 2020/204654 A1, which claims priority from and the benefit of Korean Patent Application No. 10-2019-0039098, filed on 3 Apr. 2019, the disclosures of which are incorporated herein by reference in their entireties.

### BACKGROUND

### 1. Field

The present disclosure relates to a skin treatment method using a medical laser, which has improved treatment efficacy.

### 2. Description of Related Art

A laser beam device is a device for outputting a laser beam having three characteristics of monochromaticity, coherence, and collimation, unlike regular natural light and light emitted from a lamp. A laser beam output from a laser beam device is output with energy having different wavelengths or different pulse widths according to changes such as oscillation conditions of the laser beam.

The laser beam output from such a laser beam device tends to be widely used in various industrial fields because of its excellent characteristics of monochromaticity, coherence, and collimation. For example, laser beam devices are used in various industries such as the metals industry, construction industry, shipbuilding industry, and medical industry. In particular, the usability of laser beam devices is increasing in the medical industry due to increasing treatment efficiency according to the irradiation of a laser beam.

Meanwhile, laser beam devices used in the medical industry use a laser beam having various wavelengths, pulse widths, or output energies according to various uses such as treatment purposes, cosmetic purposes, and portions to be subjected to medical treatment or cosmetic care. In particular, by repeatedly irradiating a predetermined laser beam once or several times at regular intervals, laser beams are used in treating pigment diseases such as facial capillary dilatation, facial flushing, freckles, and melasma, or removing fine wrinkles (skin aging disease) and improving skin elasticity.

However, in the treatment method using the device, when a medical laser beam is irradiated to an area deviating from a treatment area including not only a target area but also a deep area from the skin surface, unnecessary skin damage may be caused, and this may cause side effects such as retarded recovery or scarring. Therefore, there is a need for a technology capable of more effectively improving the skin condition by more easily and selectively irradiating a medical laser beam to the treatment area.

### SUMMARY

One aspect provides a method for operating a laser device for skin, comprising the steps of: a) determining the irradiation area of laser beams; and b) determining a depth of focus of the laser beams, and emitting any one laser beam selected therefrom, wherein the focus depth of the laser beams is formed between a position 5 mm inside the skin layer (−5 mm) and a position 20 mm outside the skin layer (+20 mm), on the basis of the skin surface.

Another aspect provides a treatment method for improving the condition of skin, comprising the steps of: a) determining the irradiation area of laser beams; and b) determining the focus depth of the laser beams, and emitting any one laser beam selected therefrom, wherein the focus depth of the laser beams is formed between a position 5 mm inside the skin layer (−5 mm) and a position 20 mm outside the skin layer (+20 mm), on the basis of the skin surface.

Throughout the present specification, when an element is referred to as “comprising” another element, it means other elements may be further included, but does not preclude the possibility of excluding the stated elements, unless the context clearly indicates otherwise. In addition, various steps may be performed differently from a specified order unless the context clearly indicates a specific order. That is, various steps may be performed in the same order as specified, may be performed substantially simultaneously, or may be performed in the reverse order.

One aspect is a method for operating a laser device for skin, comprising:

a) determining the irradiation area of laser beams; and

b) determining the focus depth of the laser beams, and emitting any one laser beam selected therefrom,

wherein the focus depth of the laser beams is formed between a position 5 mm inside a skin layer (−5 mm) and a position 20 mm outside the skin layer (+20 mm), on the basis of the skin surface.

As used herein, the term “laser device for skin” is an equipment or device used for skin treatment of a medical laser, and may be, for example, a medical laser device based on laser-induced optical breakdown (LIOB). The laser-induced optical breakdown refers to a phenomenon in which a laser beam having a specific energy is irradiated to the skin to form fine bubbles in the skin layer without causing a wound on the skin surface, and is widely used to improve skin conditions, such as skin condition improvement including pore improvement, scar treatment, whitening, skin regeneration, etc.

Meanwhile, the skin is composed of a stratified squamous epithelium (Epidermis), a dense connective tissue (Dermis), and a loose connective tissue (Subcutaneous tissue). Among them, the epidermis is the outermost layer of the skin, and forms a waterproof and protective film that covers the body surface, where blood vessels are not distributed, but keratinocytes, melanocytes, etc., exist. In addition, the dermis is located below the epidermis, protects the body from pressure and tension, and provides nutrients to the epidermis. Hair follicles, sweat glands, sebaceous glands, apocrine glands, lymphatic vessels, blood vessels, etc. are distributed in the dermis, and in particular, a melanin pigment is generally deposited in the area adjacent to the epidermis. In addition, the subcutaneous tissue is located under the dermis, and tissue fluid, undifferentiated connective tissue cells, fat cells, etc. are distributed therein. As described above, the skin consists of a skin layer having clearly differentiated structures, and changes in the state of the skin are induced by physiological or pathological changes in these structures. Therefore, according to the purpose of improving the skin condition, by delivering the energy of the medical laser beam to a specific area and a specific skin layer, unnecessary skin damage can be minimized, and it is possible to contribute to improving treatment efficacy. For example, in the case of deep scar treatment, the subcutaneous tissue can be targeted in consideration of the size and location of a scar, and the dermis or epidermis can be targeted for deposited pigmentation or whitening, respectively.

FIG. 1 is a flowchart of a method for operating a laser device for skin according to an embodiment, and FIG. 2 shows a step-by-step treatment area and an operating principle of a method for operating a laser device for skin according to an embodiment.

In step a), the irradiation area of laser beams is determined (S10). In this step, the irradiation area of laser beams may be adjusted based on the pre-measured distribution of a melanin pigment, the size of pores or scars, or a skin lesion site. The laser beams may be multi-spot laser beams, and in this case, any one of a spot size and a spot interval with respect to the multi-spot laser beams may be adjusted. For example, the irradiation area, etc. may be adjusted by an irradiation area adjustment unit, such as an aperture, embedded in the laser beam irradiation unit. Each spot may be formed in various shapes such as a square, a rhombus, and a circle, and specifically, in the case of a circular shape, the diameter thereof may be in a range of 2 mm to 15 mm.

In step b), the focus depth of the laser beams is determined (S20). In the above step, the range of the focus depth may be adjusted according to the purpose of improving the skin condition.

The focus depth indicates a point centrally located in the longitudinal axis direction within the beam waist according to the diffraction law for the Gaussian laser beam. Since the laser-induced optical breakdown according to the energy transfer of laser beam occurs in the vicinity of the focus depth, specifically, at a point ±1 mm from the focus depth, adjustment of the focus depth enables energy transfer to a specific skin layer, that is, laser treatment. Meanwhile, the focus depth may be adjusted according to a commonly known method, for example, the wavelength of laser beam, the energy fluence, the distance between the laser beam and the skin, the mode of pulse width, and the like, and specifically, the focus depth may be adjusted by a depth-of-focus adjustment unit embedded in the laser beam irradiation unit, the energy fluence or wavelength of the laser beam irradiated to the skin. The mode of the pulse width may be a pico mode or a nano mode. The pico mode may refer to a laser beam having a pico-second pulse width, and the nano mode may refer to a laser beam output by modulating the pico-second pulse width, for example, dividing into two parts.

Accordingly, the laser beam may be adjusted to form a depth of focus between a position of 5 mm inside the skin layer (−5 mm) and a position 20 mm outside the skin layer (+20 mm), on the basis of the skin surface. For example, according to the purpose of improving the skin condition, the laser beam may be classified into: a first laser beam having a depth of focus formed in a range of 2 mm to 4 mm (a range of −2 mm to −4 mm) inside the skin layer, on the basis of the skin surface; a second laser beam having a depth of focus formed inside the skin layer in a range of 0.3 mm to 2 mm (a range of −0.3 mm to −2 mm); and/or a third laser beam having a depth of focus formed in a range of 5 to 20 mm (a range of +5 mm to +20 mm) outside the skin layer. In addition, the first laser beam, the second laser beam, and the third laser beam may be sequentially selected, or may be arbitrarily selected alone or in combination.

In addition, in step b), the laser beam having the irradiation area and focus depth determined is emitted (S30). The laser beam may be appropriately changed according to the purpose of improving the skin condition or the like. The laser beam may be provided to have a nano-second or pico-second pulse width, have a wavelength of 530 to 1070 nm, or may be provided to have a spot of 300 to 500 pieces/cm². For example, in relation to improving treatment efficacy or forming LIOB cavities, the laser beam may have a pico-second pulse width or may be provided to have a wavelength of 532 nm, or 1064 nm.

According to an embodiment, the laser device for skin may comprise: a main body including a laser beam supply unit; and a laser beam irradiation unit connected to the main body and irradiating a laser beam to the skin of a patient, wherein the laser beam irradiation unit comprises: an irradiating area adjustment unit; and a depth-of-focus adjustment unit.

Various components for generating a pumping laser by receiving power from the outside may be installed in the main body, which may include a laser beam supply unit. A control panel for manipulating driving contents of the laser device and a display for displaying operating contents and a control panel for operating the laser device may be installed on the outer surface of the main body. In addition, known techniques known in the art may be applied. For example, a cable is installed to extend from one side of the main body, and a laser beam irradiation unit such as a handpiece may be connected to a fastening part of an end of the cable. The fastening part of the cable may be installed so as to be connected to the laser beam irradiation unit by screw coupling, and may be configured in various other coupling methods. In addition, the laser generated from the main body may be transmitted to the laser beam irradiation unit, such as a handpiece, through an articulated arm of the laser device for medical treatment. The laser beam may be controlled through a control panel of the main body or controlled through manipulation of the laser beam irradiation unit to irradiate a laser beam for treatment or examination of a human body.

The laser beam irradiation unit may be provided with a laser path in which the laser travels, and may irradiate a laser beam to the outside in a state in which it is coupled with a cable or a refractive arm to proceed with treatment. In addition, the laser beam irradiation unit may have an irradiation area adjustment unit; and a depth-of-focus adjustment unit may be installed therein. The irradiation area adjustment unit may include a convex lens, a concave lens, and the like, and can adjust a spot interval or a spot size in the same principle as an aperture of a camera. In addition, the depth-of-focus adjustment unit may adjust the position of the depth of focus formed on the skin surface or the inner layer of the skin by adjusting the distance between the laser beam and the skin surface.

Additionally, the laser beam irradiation unit may be installed therein a configuration that can be included in a conventional laser device for skin, which may include a laser output unit, and a multi-spot forming unit. The laser output unit may generate an output laser beam having a nano-second or pico-second pulse width corresponding to the laser beam supplied from the main body, or may generate an output laser beam having a wavelength of 530 to 1070 nm, for example, 532 nm, or 1064 nm. In addition, the multi-spot forming unit is a component that converts a single-spot laser beam into a multi-spot laser beam, and may include either a micro lens array (MLA) or a diffractive optical element (DOE).

Such an integrated device configuration allows an area treated with a laser beam to be easily specified, thereby contributing to improving the efficacy and efficiency of laser treatment.

The operating method may select any one treatment area from a specific area inside the skin layer from the skin surface, and if necessary, it is also possible to sequentially change the treatment area. According to one embodiment, when targeting the skin surface, a laser beam having a relatively high energy fluence, for example, 1 to 6.0 J/cm², may be used, and when targeting the inner skin layer, a laser beam having a relatively low an energy fluence, for example 1 J/cm² or less, may be used. In addition, the same effect as described above may be achieved by adjusting the mode of the laser beam, that is, the pico mode and the nano mode. In addition, in the operating method, the size of the cavity in the skin layer formed by laser beam irradiation can also be selected. According to an embodiment, a laser beam having a relatively short wavelength, for example, 532 nm, may form a small cavity compared to a laser beam having a long wavelength, for example, 1064 nm.

Another aspect is a treatment method or a processing method for improving a skin condition, comprising:

the steps of: a) determining the irradiation area of laser beams; and b) determining the focus depth of the laser beams, and emitting any one laser beam selected therefrom, wherein the focus depth of the laser beams is formed between a position 5 mm inside a skin layer (−5 mm) and a position 20 mm outside the skin layer (+20 mm), on the basis of the skin surface.

As used herein, the term “treatment” may refer to any action in which the physiological or pathological condition of the skin is improved or beneficially changed through the method.

As used herein, the term, “improvement of skin condition” refers to a series of biological changes that bring not only skin lesion improvement, such as pore improvement, removal of deposited pigmentation, scars, wrinkles or melasma, whitening, or skin regeneration, but also aesthetics to the appearance.

As used herein, the terms “laser device for skin”, and the like, are the same as described above.

FIG. 1 is a flowchart of a method for operating a laser device for skin according to an embodiment, and FIG. 2 shows a step-by-step treatment area and an operating principle of a method for operating a laser device for skin according to an embodiment. Since the treatment method for improving the skin condition employs a technical configuration in the above-described method for operating the laser device for skin, specific details related thereto are as described above.

According to the operating method or treatment method of the laser device for skin according to an aspect, by selectively transferring the energy generated from a medical laser beam to a specific treatment area, the efficacy of laser treatment can be greatly improved.

In addition, since the selective delivery of the medical laser beam to the treatment area can be implemented in one device, the laser treatment time can be shortened, and convenience can be offered to users.

### BRIEF DESCRIPTION OF DRAWINGS

FIG. 1 is a flowchart of an operating method or a treatment method for a laser device for skin according to an embodiment.

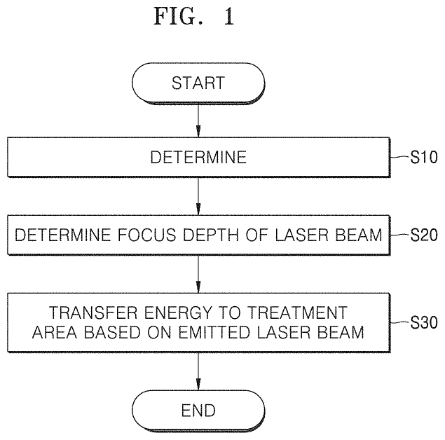

FIG. 2 shows a step-by-step treatment area and an operating principle of an operating method or treatment method for a laser device for skin according to an embodiment.

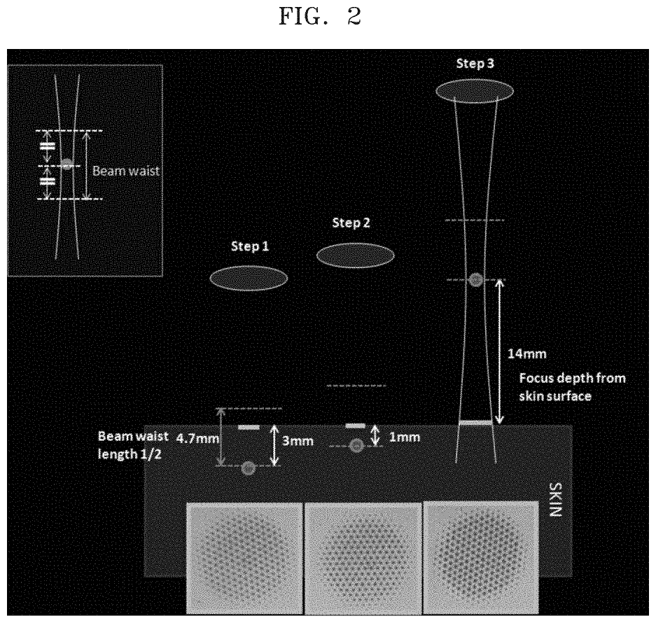

FIG. 3 shows the comparison result of evaluating skin reactivity according to focus depths (steps 1 and 2) of a laser beam according to an embodiment.

FIG. 4 is a diagram for confirming the change of the treatment area according to the energy fluence of a laser beam (1064 nm) according to an embodiment, in which: FIG. 4A shows a result of irradiating a laser beam #3 to skin; FIG. 4B shows a result of irradiating a laser beam #4 to skin; and FIG. 4C shows a result of irradiating a laser beam #5 to skin.

FIG. 5 shows a confirmation result of a change in a treatment area according to a laser beam (532 nm) according to an exemplary embodiment.

FIG. 6 shows a skin treatment result according to an embodiment, showing a change in treatment efficacy according to the energy fluence of a laser beam.

FIG. 7 shows a skin treatment result according to an embodiment, in which the treatment effect is confirmed at 4 weeks after one session of treatment.

### DETAILED DESCRIPTION

Hereinafter, the present invention will be described in more detail through examples. However, these examples are provided for illustrative purposes only, and the scope of the present invention is not limited to these examples.

### EXAMPLE 1. EVALUATION OF SKIN REACTIVITY ACCORDING TO DEPTH OF FOCUS

In this example, by using the laser device for skin according to one embodiment, after irradiating the skin with laser beams having different depths of focus, the reactivities of the skin according to the laser beams irradiated were compared. Specifically, a laser beam marked as step 1 was set to form a depth of focus in a range of 2 mm to 4 mm inside a skin layer (a range of −2 mm to −4 mm), and a laser beam marked in step 2 was set to form a depth of focus in a range of 0.3 mm to 2 mm inside the skin layer (a range of −0.3 mm to −2 mm). Thereafter, the laser beams were applied to the skin, respectively, and changes in the skin were observed with the naked eye. Meanwhile, additional experimental conditions in this example are shown in Table 1 below.

TABLE 1

| NO | Wavelength | Spot size | Fluence | R.R | Step | Mode |
|---|---|---|---|---|---|---|
| #1 | 1064 nm | 10 mm | 1.5 J/cm² | 5 Hz | 1 | PICO |
| #2 | 1064 nm | 10 mm | 1.5 J/cm² | 5 Hz | 2 | PICO |

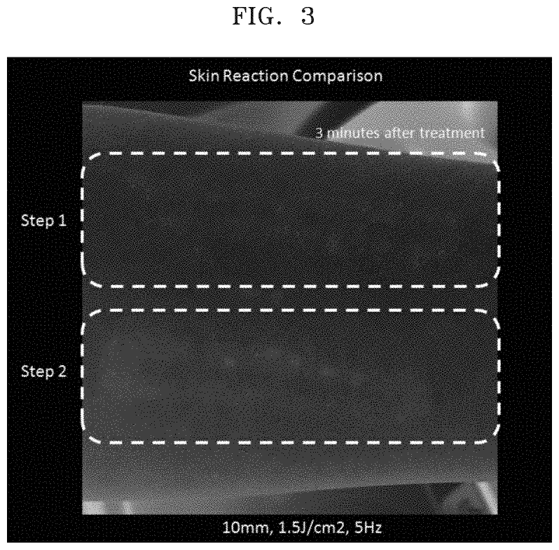

As a result, as shown in FIG. 3, it was confirmed that, even though laser beams having different depths of focus have the same irradiated area, differences in the treatment area, specifically, the target skin layer, were induced, and thus different reactivities were demonstrated in the skin different.

### EXAMPLE 2. CHANGE IN TREATMENT AREA ACCORDING TO CONDITIONS SUCH AS ENERGY FLUENCE

In this example, by using the laser device for skin according to one embodiment, after irradiating the skin with laser beams having different energy fluences, the changes in the treatment area according to the laser beams irradiated were compared. Specific steps for the laser beam are the same as in Example 1, and after the laser beam was applied to the skin, the LIOB (Laser Induced Optical Breakdown) effect was histologically evaluated. Meanwhile, additional experimental conditions in this example are shown in Table 2 below.

TABLE 2

| NO | Wavelength | Spot size | Fluence | R.R | Step | Mode |
|---|---|---|---|---|---|---|
| #3 | 1064 nm | 4 mm | 6.0 J/cm² | Single | 1 | PICO |
| #4 | 1064 nm | 4 mm | 2.8 J/cm² | Single | 1 | Nano |
| #5 | 1064 nm | 10 mm | 0.3 J/cm² | Single | 1 | PICO |

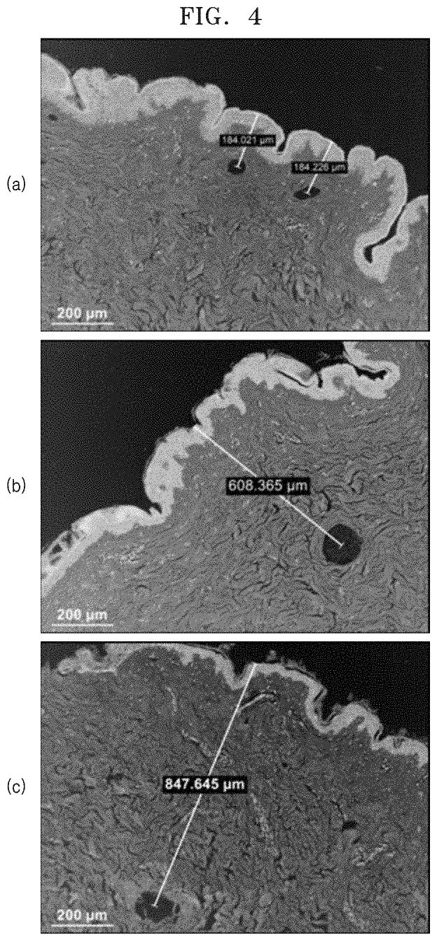

As a result, as shown in FIGS. 4A to 4C, laser beams having relatively low energy fluences (#4, #5) were able to transfer energy to deeper skin layers, compared to the laser beams having high energy fluence (#3), and the nano-mode laser beam (#4)) showed the same effect as above compared to the pico-mode laser beam (#3).

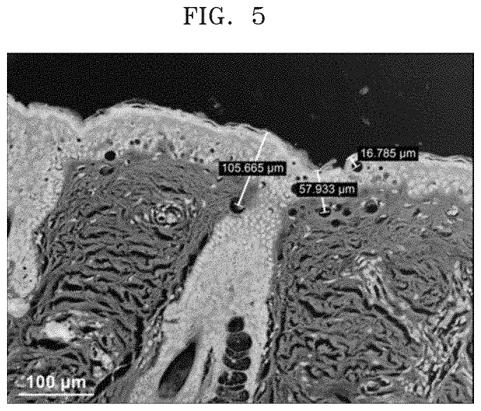

In addition, according to the conditions of Table 3 below, as a result of confirming the change in the treatment area according to the wavelength, as shown in FIG. 5, it was confirmed that a laser beam having a relatively short wavelength (532 nm) had a smaller cavity formed in the skin layer generated by the LIOB effect than a laser beam having a long wavelength (1064 nm).

TABLE 3

| NO | Wavelength | Spot size | Fluence | R.R | Step | Mode |
|---|---|---|---|---|---|---|
| #6 | 532 nm | 3.3 mm | 0.45 J/cm² | Single | 1 | PICO |

That is, according to the above conditions, it was found that the treatment area of the laser beam could be selectively adjusted.

### EXAMPLE 3. EVALUATION OF TREATMENT EFFICACY ACCORDING TO LASER BEAM CONDITIONS

In this example, by using the laser device for skin according to one embodiment, the changes in the treatment efficacy according to conditions were compared. Specific steps for the laser beam are the same as in Example 1, and additional experimental conditions are shown in Table 4 below.

TABLE 4

| Split | | Spot | | | | | |
|---|---|---|---|---|---|---|---|
| side | Wavelength | size | Fluence | R.R | Pass | Mode | Step |
| R | 1064 nm | 6 mm | 0.7 J/cm² | 5 Hz | 3 | Pico | 2 |
| L | 1064 nm | 6 mm | 1.0 J/cm² | 5 Hz | 3 | Pico | 2 |

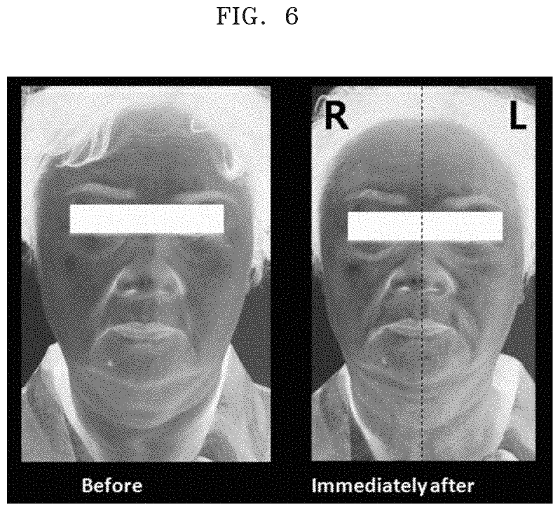

As a result, as shown in FIG. 6, in the area (L) treated with a laser beam having a high energy fluence, stronger edema and erythema appeared as the skin reaction immediately after treatment than in the area (R) treated with the laser beam having a low energy fluence, and a longer time was taken to recover to the original skin condition after treatment.

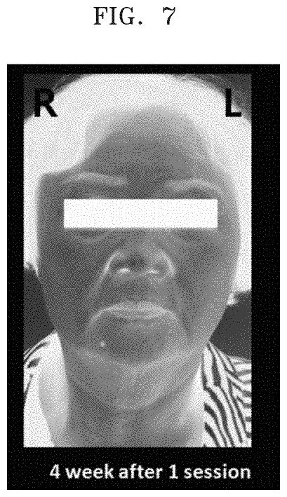

In addition, as shown in FIG. 7, as a result of confirming the treatment effect at 4 weeks after one session of treatment, the overall skin texture and skin color of the treatment area were significantly improved, compared to before treatment, and in particular, the improvement effect of the area (L) treated with a laser beam having a high energy fluence was more excellent, and the improvement effect of (L) was more excellent, and the treatment also influenced on the improvement effect of local pigmentation.

What is claimed is:

1. An operating method for a laser device for skin, the method comprising:
a) determining an irradiation area of laser beams; and
b) determining a depth of focus of the laser beams, and emitting any one laser beam selected therefrom,
wherein a focus depth of the laser beams is formed between a position 5 mm inside a skin layer (−5 mm) and a position 20 mm outside the skin layer (+20 mm), relative to a skin surface.

2. The method of claim 1, wherein the irradiation area is selected from any diameter in a range of 2 mm to 15 mm, on the basis of the skin surface.

3. The method of claim 1, wherein b) is emitting: a first laser beam having a depth of focus formed in a range of 2 mm to 4 mm (a range of −2 mm to −4 mm) inside the skin layer, on the basis of the skin surface;
a second laser beam having a depth of focus formed in a range of 0.3 mm to 2 mm (a range of −0.3 mm to −2 mm) inside the skin layer; or
a third laser beam having a depth of focus formed in a range of 5 to 20 mm (a range of +5 mm to +20 mm) outside the skin layer.

4. The method of claim 3, wherein in b), the first laser beam, the second laser beam, and the third laser beam are sequentially emitted.

5. The method of claim 1, wherein the laser beam has a nano-second or pico-second pulse width.

6. The method of claim 1, wherein the laser beam has a wavelength of 530 to 1070 nm.

7. The method of claim 1, wherein the laser beam is a multi-spot laser beam and has spots of 300 to 500 pieces/cm².

8. The method of claim 1, wherein the laser device for skin comprises:
a main body including a laser beam supply unit; and a laser beam irradiation unit which is connected to the main body and irradiates a laser beam to skin of a patient,
wherein the laser beam irradiation unit comprises: an irradiating area adjustment unit; and a depth-of-focus adjustment unit.

9. A treatment method for improving a skin condition, comprising:
a) determining an irradiation area of laser beams; and
b) determining a focus depth of the laser beams, and emitting any one laser beam selected therefrom,
wherein the focus depth of the laser beams is formed between a position 5 mm inside a skin layer (−5 mm) and a position 20 mm outside the skin layer (+20 mm), on the basis of a skin surface.

10. The treatment method of claim 9, wherein the irradiation area is selected from any diameter in a range of 2 mm to 15 mm, on the basis of the skin surface.

11. The treatment method of claim 9, wherein b) is emitting:
a first laser beam having a depth of focus formed in a range of 2 mm to 4 mm (a range of −2 mm to −4 mm) inside the skin layer, on the basis of the skin surface;
a second laser beam having a depth of focus formed in a range of 0.3 mm to 2 mm (a range of −0.3 mm to −2 mm) inside the skin layer; or
a third laser beam having a depth of focus formed in a range of 5 to 20 mm (a range of +5 mm to +20 mm) outside the skin layer.

12. The treatment method of claim 11, wherein in b), the first laser beam, the second laser beam, and the third laser beam are sequentially emitted.

13. The treatment method of claim 9, wherein the laser beam has a nano-second or pico-second pulse width.

14. The treatment method of claim 9, wherein the laser beam has a wavelength of 530 to 1070 nm.

15. The treatment method of claim 9, wherein the laser beam is a multi-spot laser beam and has spots of 300 to 500 pieces/cm².

16. The treatment method of claim 9, wherein the method is performed by a laser device for skin, the laser device comprising:
a main body including a laser beam supply unit; and a laser beam irradiation unit which is connected to the main body and irradiates a laser beam to skin of a patient,
wherein the laser beam irradiation unit comprises: an irradiating area adjustment unit; and a depth-of-focus adjustment unit.

17. The treatment method of claim 9, wherein the method is for pore improvement, removal of deposited pigmentation, scars, wrinkles or melasma, whitening, or skin regeneration.
